(19) United States
(12) Patent Application Publication
(10) Pub. No.: US 2025/0069685 A1
(43) Pub. Date: Feb. 27, 2025

(54) BIOLOGICAL INFORMATION PROCESSING METHOD AND BIOLOGICAL INFORMATION PROCESSING DEVICE

(71) Applicant: JVCKENWOOD Corporation, Yokohama-shi (JP)

(72) Inventors: Ako Suzuki, Yokohama-shi (JP); Takayuki Sugahara, Yokohama-shi (JP)

(21) Appl. No.: 18/939,581

(22) Filed: Nov. 7, 2024

Related U.S. Application Data
(63) Continuation of application No. PCT/JP2023/014051, filed on Apr. 5, 2023.

(30) Foreign Application Priority Data
May 31, 2022 (JP) 2022-088346

(51) Int. Cl. G16B 15/00; G16B 20/00

(52) U.S. Cl. CPC G16B 15/00; G16B 20/00

(57) ABSTRACT

A biological information processing method includes: acquiring differences between a chromatin structure of a living body and a biological state at a first point of time and a chromatin structure of the living body and a biological state at a second point of time after the first point of time respectively; identifying, based on the difference in the chromatin structure of the living body and the difference in the biological state, histone modification that causes alteration of the chromatin structure which alters the biological state; acquiring correspondence information indicating a correspondence between the identified histone modification and the biological state of the living body corresponding to the identified histone modification; and identifying, based on the correspondence information, the histone modification for activating or suppressing expression of a specified biological state in a target living body of a same type as the living body.

### CROSS-REFERENCE TO RELATED APPLICATIONS

This application is a Continuation of PCT International Application No. PCT/JP2023/014051 filed on Apr. 5, 2023 which claims the benefit of priority from Japanese Patent Application No. 2022-088346 filed on May 31, 2022, the entire contents of both of which are incorporated herein by reference.

### FIELD OF THE INVENTION

The present application relates to a biological information processing method and a biological information processing device.

### BACKGROUND OF THE INVENTION

The term epigenetics points to a mechanism in which, without any alteration of DNA sequence, gene expression or phenotypic alteration is inherited from cells or multicellular organisms to their progeny. It is believed that the mechanism of epigenetics involves chemical modification of DNA such as DHA methylation, chemical modification of histone, and alteration of structure or stability of nucleosomes or chromatin based on such chemical modification.

Japanese Patent Application Laid-open No. 2018-042560 discloses a method in which information about history of epigenetic modification in a living body is retrieved, similarity with another living body is analyzed for the retrieved information, and future alteration of a state of the living body is estimated and controlled.

Japanese Patent Application Laid-open No. 2018-042560 does not suggest acquiring information about activation and suppression of expression of a biological state in a living body by using epigenetic modification.

### SUMMARY OF THE INVENTION

A biological information processing method and a biological information processing device are disclosed.

According to one aspect of the present application, there is provided a biological information processing method comprising: acquiring differences between a chromatin structure of a living body and a biological state at a first point of time and a chromatin structure of the living body and a biological state at a second point of time after the first point of time respectively; identifying, based on the difference in the chromatin structure of the living body and the difference in the biological state, histone modification that causes alteration of the chromatin structure which alters the biological state; acquiring correspondence information indicating a correspondence between the identified histone modification and the biological state of the living body corresponding to the identified histone modification; and identifying, based on the correspondence information, the histone modification for activating or suppressing expression of a specified biological state in a target living body of a same type as the living body.

According to one aspect of the present application, there is provided a biological information processing device comprising: an arithmetic unit configured to: acquire differences between a chromatin structure of a living body and a biological state at a first point of time and a chromatin structure of the living body and a biological state at a second point of time after the first point of time respectively, identify, based on the difference in the chromatin structure of the living body and the difference in the biological state, histone modification that causes alteration of the chromatin structure which alters the biological state, and acquire correspondence information indicating a correspondence between the identified histone modification and the biological state of the living body corresponding to the identified histone modification; and an acquisition unit configured to acquire specification information for activating or suppressing expression of a specified biological state in a target living body of a same type as the living body, wherein the arithmetic unit is further configured to identify, based on the correspondence information and the specification information, the histone modification corresponding to the biological state specified in the specification information.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a flowchart for explaining a flow of a biological information processing method according to a present embodiment;

FIG. 2 is a diagram schematically illustrating a principle of epigenetics;

FIG. 3 is a diagram illustrating an example of a neural network; and

FIG. 4 is a functional block diagram illustrating an example of a biological information processing device according to the present embodiment.

### DETAILED DESCRIPTION OF THE INVENTION

An exemplary embodiment of a biological information processing method and a biological information processing device according to the present application is described below in detail. However, the present invention is not limited by the embodiment described below. Moreover, the constituent elements according to the embodiment described below include constituent elements that are simple and that can be replaced with other constituent elements by a person skilled in the art, or include practically identical constituent elements.

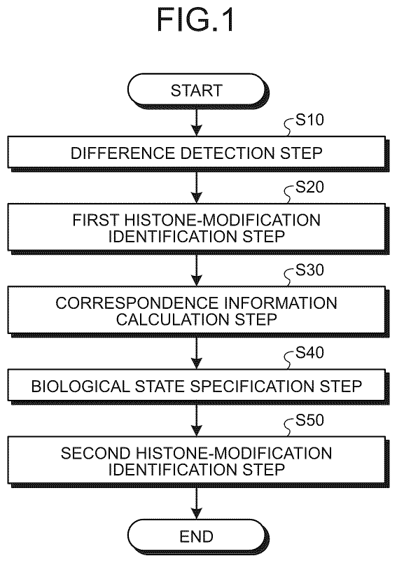

FIG. 1 is a flowchart for explaining a flow of a biological information processing method according to the present embodiment. As illustrated in FIG. 1, the biological information processing method according to the present embodiment includes a difference detection step (S10), a first histone-modification identification step (S20), a correspondence information calculation step (S30), a biological state specification step (S40), and a second histone-modification identification step (S50).

Firstly, an explanation is given about a principle of the biological information processing method according to the present embodiment. The biological information processing method according to the present embodiment is based on a mechanism called epigenetics in which, without any alteration of DNA sequence, gene expression or phenotypic alteration is inherited from cells or multicellular organisms to their progeny.

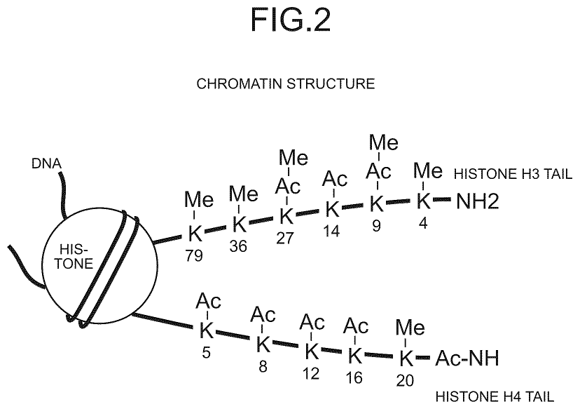

FIG. 2 is a diagram that schematically illustrates a principle of epigenetics, and DNA exhibits a chromatin structure wound around a protein called histone. When histone is modified by methylation, ubiquitination, phosphorylation, or acetylation, alteration occurs in chromatin structure. As a result of the alteration occurring in the chromatin structure, there occurs alteration of activation and suppression of gene-based expression of a biological state.

For example, gene-based expression of a biological state is suppressed by methylation of histone. Histone is formed with, for example, 20 to 30 amino acids and has a tail-like shape. The amino acids constituting histone become a target of various types of modification. As illustrated in FIG. 2, when a histone H3 tail is modified with methylation (Me) and a histone H4 tail is modified with acetylation (Ac), there occurs alteration of activation and suppression of gene-based expression of a biological state. More particularly, when amino acids indicated by K4, K36, and K79 in histone H3 tail are methylated, there is activation of transcription for copying gene information from DNA to mRNA. Moreover, when amino acids indicated by K9 and K27 in the histone H3 tail and an amino acid indicated by K20 in the histone H4 tail are methylated, transcription is suppressed.

In this way, by alteration occurring in histone modification, gene-based expression of a biological state is activated or suppressed.

A person of the present application found out that alteration of the chromatin structure is likely to be caused by, for example, predetermined factors. Examples of the predetermined factor include living environment factors such as the latitude, the longitude, and the climate of a place where a living body lives; experience factors based on experiences gained by the living body in life; psychological factors based on psychological stress and happiness of the living body; age factors based on an age of the living body; and gender factors based on gender of the living body.

For example, it is a known fact that a living body that has lived in an environment in low altitude and high temperature for a long period of time has high perspirability. In that case, as a result of being exposed to a high-temperature environment for a long period of time, it is possible to think that chromatin structure of a living body has altered such that gene-based expression of perspirability is activated.

For example, in a case of a human being, a brain of an infant child is in a process of growing, and large-scale and complex brain tissues like a brain of an adult are yet to be fully formed. In a human brain, a long-term memory acquired up to a predetermined age such as up to about three years after birth represents unstable memory created by an undeveloped brain and gets lost due to aging. The brain grows such that new brain cells add to the existing brain cells instead of the old brain cells getting replaced by new brain cells. It indicates that, although the old memory is not cleared out due to neural development, the addition of new cells results in reconfiguration of the memory inside the brain, and the old memory becomes inaccessible. That is, in the case of a human being, by the growth of the brain for about three years after birth, it is possible to think that the chromatin structure alters such that the memory stored in the old cells of the brain becomes inaccessible.

In the example described above, for example, when a living body having low perspirability has to live in a high-temperature environment, the living body can adopt to the high-temperature environment if the perspirability can be enhanced. In a brain of a human being, capability of instinctive behavior or feeling geomagnetism may be left in a memory stored in the pre-reconfiguration old cells. If such capability can be utilized after growth, it may lead to new discoveries. Meanwhile, aside from the examples described above, in a case of having genes which are more apt to develop cancer, diabetes, or autoimmune disease, it is believed that estimating histone modification for suppressing such expression would prove useful.

In that regard, in the present embodiment, regarding a living body, differences are acquired between the chromatin structure and the biological state at a first point of time and the chromatin structure and the biological state at a second point of time after the first point of time respectively. Then, based on the difference in the chromatin structure and the difference in the biological state of the living body, histone modification is identified which causes alteration of the chromatin structure that alters the biological state; and correspondence information is acquired that indicates a correspondence between the identified histone modification and the biological state of the living body corresponding to the histone modification. Subsequently, based on the correspondence information, histone modification is estimated which would enable activation or suppression of the expression of a specified biological state in a target living body of the same type as the concerned living body.

Firstly, at the difference detection step (S10), regarding a living body, differences between the chromatin structure and the biological state at the first point of time and the chromatin structure and the biological state at the second point of time after the first point of time are acquired respectively. The chromatin structure of a living body can be acquired by, for example, collecting cells of the living body and analyzing DNA of the collected cells according to a known method. In the present embodiment, biological information contains information related to the chromatin structure of the living body. Moreover, the biological state includes a state of the living body, such as capability of the living body or development of disease, which alters due to the alteration of the chromatin structure. A wearable device capable of acquiring the biological state of the living body can be attached to the living body for example, and the biological state can be acquired from the living body at predetermined timings. Examples of the biological state include a walking speed, a running speed, a muscle mass, a heart rate, a respiratory rate, a blood pressure, a body temperature, brain waves, a brain blood flow, an amount of perspiration, and presence or absence of cancer cells.

The first point of time and the second point of time can be set in an arbitrary manner. In the case in which the living body is a human being and in which the same individual is present at the first point of time and at the second point of time, the first point of time can be, for example, a point of time before a predetermined age such as about three years after birth, and the second point of time can be a point of time after the predetermined age such as about three years after birth. In that case, it becomes possible to appropriately detect the alteration occurring in the chromatin structure before and after inaccessibility to the memory stored in the old cells inside the brain at the predetermined age such as about three years after birth. Meanwhile, the living body can be some other living organism such as an animal or a plant other than a human being.

The living body in which the difference in the chromatin structure is detected can be the same individual as the target living body (explained later) or can be an individual of a previous generation than the target individual. Conventionally, it was believed that acquired influences attributed to the environment are not inherited to the next generation. For example, smoking causes alteration of composition of the lung cells and causes cancer. It was believed that such acquired influence is not inherited across generations. That is, it was assumed that the epigenetic memory is completely cleared during division of egg cells. However, in recent years, it has been demonstrated that there is a possibility that the acquired influences are actually inherited across generations.

Regarding a manner in which acquired influences are genetically inherited, for example, in a human being, it is now known that H3K23me3 is related to the suppression of the genetic expression that causes alteration of the chromatin structure representing packaging of DNA inside the cell nuclei. It was discovered that, even if other epigenetic modifications are eliminated, the modification attributed to H3K27me3 remains present in an embryo after fertilization. It implies that a trace of the acquired influences attributed to the environment are inherited from the mother to her child. The inherited epigenetic modification is important to embryonic development. For example, it is known that, if a trigger enzyme for altering the NA packaging of H3K25me3 is removed, an embryo lacking H3K27me3 in an early stage of the embryonic development is destroyed before completion of the embryonic development. That is, in reproduction, the epigenetic information not only is inherited from generation to generation but is also important to the development of the embryo.

It is a known fact that if a number of important developmental genes that are usually turned off during an initial stage of the embryonic development are turned on in an embryo lacking H3K27me3 and those genes are activated too early during the division of the egg cells, the embryonic development is destroyed. Thus, in order to ensure processing and accurate transcription of a genetic code of the embryo, the inherited epigenetic information is required.

These facts indicate that a human being inherits properties beyond genes (such as a fine-tuned and important gene regulatory mechanism that is likely to affect environment and an individual lifestyle). That serves as a basis for the fact that environmental adaptation acquired by a human being during a process of survival is inherited to progeny via germline. On the other hand, a collapse of the epigenetic mechanism is likely to cause diseases such as cancer, diabetes, and autoimmune diseases.

In the first histone-modification identification step (S20), histone modification is identified that causes alteration of the chromatin structure which results in alteration of the biological state based on the difference in the chromatin structure and the difference of the biological state of the living body detected at the difference detection step (S10).

At the correspondence information calculation step (S30), correspondence information is acquired that indicates the correspondence between the identified histone modification and the biological state of the living body corresponding to the identified histone modification.

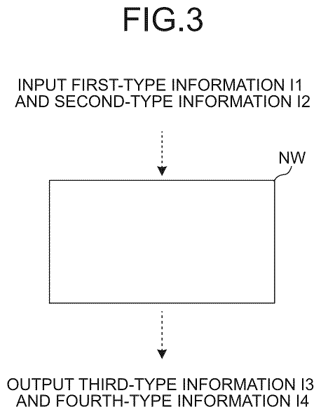

For example, a neural network can be generated in advance in which, when the chromatin structure and the biological information of the living body at the first point of time and the chromatin structure and the biological information of the living body at the second point of time are input, the histone modification that would cause alteration of the chromatin structure which would alters the biological state can be calculated and output based on the input result, and which associates the output result with the biological information. FIG. 3 is a diagram illustrating an example of a neural network. A neural network NW illustrated in FIG. 3 receives input of first-type information I1 containing the chromatin structure and the biological information at the first point of time, and receives input of second-type information I2 containing the chromatin structure and the biological information at the second point of time, outputs third-type information I3 containing the altered biological state, and outputs fourth-type information I4 containing the histone modification that would cause alteration of the chromatin structure which would alters the biological state, and associates the third-type information I3 with the fourth-type type information I4. Thus, as a result of inputting the first-type information I1 and the second-type information I2 to the neural network NW, the correspondence information can be acquired in which the third-type information I3 is associated with the fourth-type information I4. Hence, the difference detection step (S10), the first histone-modification identification step (S20), and the correspondence information calculation step (S30) can be performed immediately.

At the biological state specification step (S40), for the target living body, a target biological state and information indicating whether the expression of the target biological state is to be activated or suppressed are specified. Herein, the target living body is of the same type as the living body for which the differences are detected at the difference detection step (S10).

At the second histone-modification identification step (S50), based on the correspondence information calculated at the correspondence information calculation step (S30), histone modification is identified which would activate or suppress the expression of the biological state in the target living body which is specified at the biological state specification step (S40).

When the living body present at the first point of time is an individual of a previous generation of the living body present at the second point of time, the difference between the predetermined factors of the living body at the first point of time and the predetermined factors of the living body at the second point of time can be further acquired either independently from the steps S10 to S50 explained above or in addition to the steps S10 to S50 explained above. In that case, based on the differences in the chromatin structure and the predetermined factors of the living body, predetermined factors that would cause alteration of the chromatin structure of the living body can be identified, and correspondence information of the previous generation that indicates the correspondence between the identified predetermined factors and the chromatin structure can be acquired. Then, for example, based on the correspondence information of the previous generation, the alteration of the chromatin structure of the target living body can be estimated. For example, assume that a living body in the parent generation gets cancer at the age of 20 years, the chromatin structure of that living body at the age of 19 years is different from that at the age of 20 years, and predetermined factors such as the living environment factors including the living environment and the customs and the psychological factors at the age of 19 years are different from those at the age of 20 years. In that case, in a living body in a child generation, probability of getting cancer can be reduced by avoiding the same living environment, the same customs, and the same psychological state.

Moreover, for example, assume that the living body is a human being, the same individual is present at the first point of time and the second point of time, the first point of time is a point of time before the age of about three years after birth representing the predetermined age, and the second point of time is a point of time after the age of about three years after birth representing the predetermined age. In that case, as a result of performing the operations at the steps S10 to S30 explained above, it becomes possible to detect the alteration of the chromatin structure before and after occurring alteration in the biological state in which the memory stored in the old cells inside the brain at the age of about three years after birth is unable to be accessed; to identify the histone modification that causes the alteration of the chromatin structure; and to appropriately acquire the correspondence information between the identified histone modification and the concerned biological state. In that case, at the biological state specification step (S40), for example, if the biological state (capability) of accessing the memory stored in the old cells inside the brain is specified, the histone modification corresponding to the specified biological state can be appropriately identified based on the correspondence information. Thus, by altering the chromatin structure of the target living body based on the identified histone modification, it becomes possible to express and activate the biological state of accessing the memory stored in the old cells inside the brain of the target living body. Meanwhile, although the age of about three years after birth is treated as the predetermined age as an example, that is not the only possible example. That is, the predetermined age can be set to an arbitrary age.

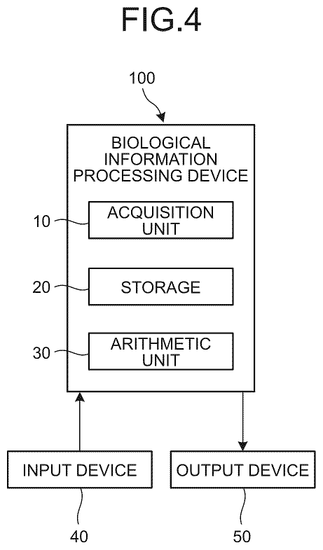

FIG. 4 is a functional block diagram illustrating an example of a biological information processing device 100 according to the present embodiment. As illustrated in FIG. 4, the biological information processing device 100 includes a processing device such as a CPU (Central Processing Unit) and a storage device such as a RAM (Random Access Memory) or a ROM (Read Only Memory).

The biological information processing device 100 includes an acquisition unit 10, a storage 20, and an arithmetic unit 30. Moreover, the biological information processing device 100 can also include a communication unit (not illustrated). The biological information processing device 100 is connected to external devices such as an input device 40 and an output device 50. The input device 40 receives input of various types of operations. The input device 40 is configured using, for example, various types of input devices such as a keyboard, a mouse, buttons, switches, and a touch-sensitive panel. For example, the input device 40 can input, to the biological information processing device 100, the target biological state and the information indicating whether to activate or suppress the expression of the biological state. The output device 50 is configured using a display and a voice output unit. The output device 50 outputs the processing details of the biological information processing device 100 in a form of texts, videos, and audios. The acquisition unit 10 acquires a variety of information. The acquisition unit 10 acquires, for example the information input by the input device 40. Moreover, the acquisition unit 10 acquires the information received by the communication unit (not illustrated).

The storage 20 is used to store a variety of information. The storage 20 includes a storage device such as a hard disk drive or a solid state drive. Meanwhile, as the storage 20, it is also possible to use an external memory medium such as a removable disk. In the storage 20 a variety of data and applications, such as the neural network explained earlier and the correspondence information output in the neural network is stored.

The arithmetic unit 30 performs a variety of computing. The arithmetic unit 30 performs an operation corresponding to the second histone-modification identification step (S50). Moreover, when the neural network explained earlier is stored in the storage 20, the arithmetic unit 30 is able to use the neural network and perform operations corresponding to the difference detection step (S10), the first histone-modification identification step (S20), and the correspondence information calculation step (S30). Meanwhile, the operations corresponding to the difference detection step (S10), the first histone-modification identification step (S20), and the correspondence information calculation step (S30) can alternatively be performed in some other device other than the biological information processing device 100. In that case, the correspondence information acquired in advance in the other device can be stored in the storage 20.

As explained above, in the biological information processing method according to the present embodiment, regarding a living body, the differences are acquired between the chromatin structure of the living body and the biological state at the first point of time and the chromatin structure of the living body and the biological state at the second point of time after the first point of time respectively. Then, based on the difference in the chromatin structure of the living body and the difference in the biological state, histone modification is identified which causes alteration of the chromatin structure that alters the biological state; and correspondence information is acquired that indicates the correspondence between the identified histone modification and the biological state of the living body corresponding to the histone modification. Subsequently, based on the correspondence information, histone modification is estimated which would enable activation or suppression of the expression of a specified biological state in the target living body that is of the same type as the concerned living body.

The biological information processing device 100 according to the present embodiment includes the arithmetic unit 30 that acquires the differences between the chromatin structure of the living body and the biological state at the first point of time and the chromatin structure of the living body and the biological state at the second point of time after the first point of time respectively; identifies, based on the difference in the chromatin structure of the living body and the difference in the biological state, histone modification which causes alteration of the chromatin structure that alters the biological state; and acquires correspondence information that indicates the correspondence between the identified histone modification and the biological state of the living body corresponding to the histone modification. Moreover, the biological information processing device 100 according to the present embodiment includes the acquisition unit 10 that acquires specification information for enabling activation or suppression of the expression of the biological state in the target living body that is of the same type as the concerned living body. Based on the correspondence information and the specification information, the arithmetic unit 30 identifies the histone modification corresponding to the biological state specified in the specification information.

According to such a configuration, regarding a living body, the differences are acquired between the chromatin structure of the living body and the biological state at the first point of time and the chromatin structure of the living body and the biological state at the second point of time after the first point of time; and based on the difference in the chromatin structure of the living body and the difference in the biological state, histone modification is identified which causes alteration of the chromatin structure that alters the biological state. Thus, the histone modification can be identified with high accuracy. Moreover, based on the correspondence information indicating the correspondence between the identified histone modification and the biological state, it becomes possible to appropriately identify the histone modification for activating or suppressing the expression of the biological state in the target living body. As a result, it becomes possible to appropriately acquire the information related to the activation and the suppression of the expression of the biological state in the living body. By changing the chromatin structure of the target living body based on the histone modification estimated as explained above, it becomes possible to activate or suppress the expression of a predetermined biological state in the target living body.

In the biological information processing method according to the present embodiment, the living body at the first point of time is an individual of a previous generation of the living body at the second point of time, the difference between predetermined factors of the living body at the first point of time and predetermined factors of the living body at the second point of time is further acquired. Then, based on the difference in the chromatin structure and the predetermined factors of the living body, the predetermined factor causing alteration of the chromatin structure of the living body is identified; correspondence information of the previous generation is acquired that indicates the correspondence between the identified predetermined factor and the chromatin structure; and, based on the correspondence information of the previous generation, the alteration of the chromatin structure of the target living body is estimated. According to such a configuration, the histone modification occurring due to a predetermined factor can be identified with high accuracy. Moreover, based on the difference for the individual of the previous generation of the target living body, it becomes possible to appropriately acquire the information related to the activation and the suppression of the expression of the biological state for an individual of the subsequent generation. Moreover, since the alteration of the histone modification attributed to the alteration of the chromatin structure in the parent generation can be figured out before a child is born, it becomes possible to take measures in the child generation, such as artificially performing histone modification within a specified time period. Furthermore, for example, in the living body of the parent generation, when the chromatin structure at the first point of time is different from the chromatin structure at the second point of time and also the predetermined factors at the first point of time are different from the predetermined factors at the second point of time, the expression of specific biological information can either be activated or be suppressed for the living body in the child generation by avoiding those predetermined factors in the life.

In the biological information processing method according to the present embodiment, the same individual is treated as the living body. The first point of time is a point of time before the predetermined age, and the second point of time is a point of time after the predetermined age. With such a configuration, for the same individual, the differences in the histone modification of the chromatin structure which alters before and after the predetermined age are associated and a correlation thereof is acquired, so that it becomes possible to appropriately acquire the histone modification for activating the expression of the biological state that is obtained till the predetermined age.

Herein, although the present application is described with reference to the abovementioned embodiment for a complete and clear disclosure, the appended claims are not to be thus limited but are to be construed as embodying all modifications and alternative constructions that may occur to one skilled in the art that fairly fall within the basic teaching herein set forth. For example, in the embodiment described above, the explanation is given about the example in which the difference between predetermined factors of the living body at the first point of time and predetermined factors of the living body at the second point of time is further acquired. Then, based on the differences in the chromatin structure and the predetermined factors of the living body, the predetermined factor causing alteration of the chromatin structure of the living body is identified; correspondence information of the previous generation is acquired that indicates the correspondence between the identified predetermined factor and the chromatin structure; and, based on the correspondence information of the previous generation, the alteration of the chromatin structure of the target living body is estimated. However, that is not the only possible example. Thus, in the case of acquiring the correspondence information, it is possible to consider some other factors other than the living environment factors; the psychological factors; the age factors; and the gender factors cited as the predetermined factors.

The biological information processing method and the biological information processing device according to the present application can be used in a processing device like a computer for example.

According to the present application, it becomes possible to acquire information related to activation and suppression of expression of a biological state in a living body.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A biological information processing method comprising:
acquiring differences between a chromatin structure of a living body and a biological state at a first point of time and a chromatin structure of the living body and a biological state at a second point of time after the first point of time respectively;
identifying, based on the difference in the chromatin structure of the living body and the difference in the biological state, histone modification that causes alteration of the chromatin structure which alters the biological state;
acquiring correspondence information indicating a correspondence between the identified histone modification and the biological state of the living body corresponding to the identified histone modification; and
identifying, based on the correspondence information, the histone modification for activating or suppressing expression of a specified biological state in a target living body of a same type as the living body.

2. The biological information processing method according to claim 1, wherein
the living body at the first point of time is an individual of a previous generation of the living body at the second point of time, and
the biological information processing method further comprises:
acquiring differences between predetermined factors of the living body at the first point of time and the predetermined factors of the living body at the second point of time,
identifying, based on the difference in the chromatin structure and the differences in the predetermined factors of the living body, the predetermined factor that alters the chromatin structure of the living body,
acquiring correspondence information of the previous generation that indicates a correspondence between the identified predetermined factor and the chromatin structure, and
estimating, based on the correspondence information of the previous generation, alteration of the chromatin structure of the target living body.

3. The biological information processing method according to claim 1, wherein
the living body represents a same individual,
the first point of time is a point of time before a predetermined age, and
the second point of time is a point of time after the predetermined age.

4. A biological information processing device comprising:
an arithmetic unit configured to:
acquire differences between a chromatin structure of a living body and a biological state at a first point of time and a chromatin structure of the living body and a biological state at a second point of time after the first point of time respectively,
identify, based on the difference in the chromatin structure of the living body and the difference in the biological state, histone modification that causes alteration of the chromatin structure which alters the biological state, and
acquire correspondence information indicating a correspondence between the identified histone modification and the biological state of the living body corresponding to the identified histone modification; and
an acquisition unit configured to acquire specification information for activating or suppressing expression of a specified biological state in a target living body of a same type as the living body, wherein
the arithmetic unit is further configured to identify, based on the correspondence information and the specification information, the histone modification corresponding to the biological state specified in the specification information.
